(19) United States
(12) Patent Application Publication
(10) Pub. No.: US 2023/0068937 A1
(43) Pub. Date: Mar. 2, 2023

(54) APPLICATION OF PATHOGENICITY MODEL AND TRAINING THEREOF

(71) Applicant: CONGENICA LTD., Cambridge (GB)

(72) Inventors: Sandro Morganella, Upper Cambourne (GB); Yacine Dahman, Cambridge (GB); Laura Ponting, Cambridge (GB); Emily Mackay, Cambridge (GB)

(21) Appl. No.: 17/792,521

(22) PCT Filed: Jan. 15, 2021
(86) PCT No.: PCT/GB2021/050086

§ 371 Date: Jul. 13, 2022

(30) Foreign Application Priority Data
Jan. 16, 2020 (GB) 2000649.0
Aug. 26, 2020 (GB) 2013386.4
Aug. 26, 2020 (GB) 2013387.2

(51) Int. Cl. G16B 40/00; G16B 20/20; G16B 45/00

(52) U.S. Cl. CPC G16B 40/00; G16B 20/20; G16B 45/00

(57) ABSTRACT

A computer-implemented method that is for assessing pathogenicity of a variant for a patient. Receive a variant. Determine at least one probability for the variant in relation to pathogenic metrics based on a collection of learned variants. The pathogenic metrics comprise a data representation of at least one genetic condition cluster for determining at least one probability for the variant. The combined representation of at least one probability of the variant for the patient is outputted.

The present application relates to a system, apparatus and method(s) for assessing the pathogenicity of a variant for a patient and the training of a model for the assessment thereof.

### BACKGROUND

Advancements in medical and computational technologies have enabled the analysis of genomic sequencing of biological samples based on phenotypic attributes. Genomic analysis for predicting disease-causing DNA mutations based on these attributes has been a robust area of research and development. Much uncertainty remains with these predictions due to the inherent complexity of genomic data and the abundance of noise. For instance, the complexity may be attributed to mutations that range from single nucleotide variants (SNV) to large and complex rearrangements, notwithstanding the noise during the sequencing process. The uncertainty in the prediction of these mutations poses a challenge for existing technologies or computational tools, which are inefficient and inaccurate, especially for analysing a particular variant or mutation.

Though, several computational tools have been developed for genomic data analysis and interpretation to obtain insights on genetic variants. However, these tools require extensive training of their underlying models using a large amount of labelled and/or un-labelled training data to operate the embedded machine learning algorithms, which has length run-time and is thereby resource-intensive. For example, conventional machine learning or artificial intelligence models undergo complete retraining when a new input related to a previous input of a subject is fed into such models, which is undesirable provided that diagnostic test results and other information related to a subject typically are not readily available, and usually obtain only when the diagnostic tests are conducted and when additional data related to a patient is available. Thus, the retraining of conventional models in such cases not only creates a time lag in the assessment of genomic data relating to a subject, but also increases uncertainty in the genomic interpretation, with an associated risk of misinterpretation. In the above example, a time lag can occur between a given patient's blood samples being sequenced and there arising a discovery of new relevant scientific information potentially some years afterwards; the new relevant scientific information concerns what a particular gene does when expressed. As a result of the time lag, a medical record for the given patient may potentially be marked as “unresolved” and the given patient's record not revisited later when more information becomes available.

Therefore, in light of the foregoing discussion, there exists a need to overcome the aforementioned drawbacks associated with conventional methods for processing, analyzing, or interpreting genomic data, to reduce effects of noise and to prevent over-fitting. More specifically, there is a need for a process to handle copious amounts of complex genomic data which is inherently complex to order to accurately assess a variant or mutations in the patient's biological sequences in terms of the variant's pathogenicity.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides an algorithmic framework enabling the identification of causative DNA mutations given the genomic profile and the specific phenotypic attributes of a patient.

In a first aspect, the present disclosure provides a computer-implemented method for assessing pathogenicity of a variant for a patient comprising: receiving a variant; determining at least one probability for the variant in relation to pathogenic metrics based on a collection of learned variants, wherein the pathogenic metrics comprise a data representation of at least one genetic condition cluster for determining the at least one probability for the variant; and outputting a combined representation of the at least one probability of the variant for the patient.

In a second aspect, the present disclosure provides a computer-implemented method for generating at least one genetic condition cluster for determining at least one probability of a variant in relation to pathogenic metrics comprising: receiving annotated data of at least one patient associated with a collection of variants, wherein the annotated data comprise interpretation information with associated observations corresponding to the pathogenic metrics; determining a data representation for the annotated data of at least one patient, wherein the data representation is derived using one or more generative models; and generating the at least one genetic condition cluster based on the data representation.

In a third aspect, the present disclosure provides a computer-implemented method for assessing pathogenicity of an unknown variant for a patient using a set of side information comprising: receiving the unknown variant, wherein the unknown variant is not identified in the collection of learned variants; using the set of side information corresponding to each of a subset of the collection of learned variants to train a supervised learning framework; and assessing the pathogenicity of the unknown variant based on the trained supervised learning framework.

In a fourth aspect, the present disclosure provides an apparatus for determining pathogenicity of a variant for a patient, the apparatus comprising: an input component configured to receive the variant; a processing component configured to determine whether the variant is within a collection of learned variants; a prediction component, in response to a determination that the variant is present in the collection of the learned variant, configured to generate at least one probability for the variant in relation to pathogenic metrics, wherein the pathogenic metrics comprise a data representation of at least one genetic condition cluster for determining the at least one probability for the variant; and a display component configured to display the at least one probability for the variant with respect to the pathogenic metrics, wherein the at least one probability is normalised.

In a firth aspect, the present disclosure provides a computer-implemented method for determining a probability distribution of pathogenicity for an unknown gene variant using a set of side information, the method comprising: receiving the unknown variant of a patient, wherein the unknown variant is not identified in or new to the collection of learned variants associated with a plurality of patients; assessing the pathogenicity of the unknown gene variant by using a supervised learning framework based on the set of side information; and determining the probability distribution of pathogenicity based on the assessment.

The methods described herein may be performed by software in machine readable form on a tangible or a non-transitory storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a flow diagram illustrating an example of assessing pathogenicity of a variant for a patient according to the invention;

FIG. 1b is a schematic diagram illustrating an example where the pathogenicity of a variant for a patient is assessed in relation to phenotypic and side information according to the invention;

FIG. 2a is a flow diagram illustrating an example of generating genetic condition clusters for determining at least one probability of a variant in relation to pathogenic metrics according to the invention;

FIG. 2b is a schematic diagram of an example of genetic condition clusters for determining a probability of a variant according to the invention;

FIG. 3 is a flow diagram illustrating an example of assessing pathogenicity of an unknown variant for a patient using a set of side information according to the invention;

FIG. 4 is a schematic diagram illustrating an example of genetic condition clusters extracted from annotated data to predict probabilities of the variant given the pathogenic metrics according to the invention.

FIG. 5 is a schematic diagram of a computer system suitable for implementing embodiments of the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The inventors propose a process for assessing or predicting the pathogenicity of a particular variant (e.g. a gene variant) for a patient of interest. The process utilizes at least one predictive model that is trained using annotated training data of phenotypic and/or interpretation information, which is compiled to derive a set of latent variables, in order to make the suitable assessment or prediction. In turn, the set of latent variables could be perceived as data representations of (hidden) genetic condition clusters. The genetic condition clusters are adapted to determine a set of probabilities for the variant based on a collection of variants learned by the model. The probabilities are evaluated in terms of a pathogenic metrics, where each metric ascribes to one probability determined. The combined representation of the set of probabilities are outputting to a user via the computing interface or device. Thus, the likelihood of whether the input variant is pathogenic (e.g. begin or pathogenic) or its pathogenicity can be determined by or considered in accordance with the outputted probability.

This process may iterate, and the predictive model may continue to increment with the influx of more input of phenotypic and/or interpretation information. The phenotypic and/or interpretation information comprises data points associated with patients, variants and corresponding observations from past patient interpretations embodied as a multi-dimensional data matrix. The data points may be highly sparse with respect to the size of the matrix in that the observations of the data matrix are approximately 99.96% absent. This is due at least to the size of the variant pool and the limited availability of observations associated with each variant. Nevertheless, the process herein described as the method, system, medium or apparatus presents at least a solution for overcoming the dilemma of data sparsity through the application of genetic condition clusters. In effect, the genetic condition clusters, in the abstract, map the variant to its underlying pathogenicity to the extent of solving the objective problem of data sparsity amongst other technical problems described herein.

Pathogenicity herein refers to the property of causing a particular disease. Pathogenicity of a variant is the ability of the variant in causing the disease. Pathogenicity of a variant is both a qualitative and quantitative evaluation of the variant and the likelihood for the variant and contribution to the causation of the disease. The likelihood of a variant being pathogenic may be presented as probabilities. These probabilities are associated with the variant and provide the quantitative evaluation of the variant in terms of its pathogenicity.

A variant is a mutation in genetic (DNA) sequences and transcripts (RNA) thereof, which include gene variants or other sequence mutations. In particular, the gene variants refer to single-nucleotide polymorphism (SNP), copy number variant (CNV), gene rearrangement, indels, and the like. In general, a patient with a variant may have a condition or illness caused by disease to the extent that the patient inherits SNP or mutation in the genomic DNA. Such a patient may have one or more variant that includes, but are not limited to, for example, copy number variants (CNVs), indels, single nucleotide variants (SNV), and other mutations responsible for genetic diseases. As such, a variant is any difference, in genomic DNA, between the healthy individual and the patient in the context of genetic screening.

For example, gene ‘X’ may have two variants: ‘A’ and ‘B’. Both ‘A’ and ‘B’ variants are located at different loci of the gene ‘X’ and are responsible for disease ‘D’. Provided that a certain DNA mutation (e.g. where expected ‘A’ nucleotide is replaced by ‘C’ nucleotide) when present at specific coding regions of the gene makes such gene potentially pathogenetic, the presence this stretch of DNA at the locus of variant ‘A’ can be readily associated variant ‘A’ with disease ‘D’ for a new patient as opposed to variant ‘B’ that does not exhibit the same DNA sequence. The variants associated with gene ‘X’ and their corresponding relation to disease ‘D’ may be adapted to the model described in the following sections and as learned variants of the method, system, medium or apparatus herein described.

Further, it is found that a certain example stretch of a gene (e.g. ‘AAAAATAAAAAT’) when present as variants at specific coding regions of the gene (e.g. ‘AA’ to ‘CC’) makes the gene potentially pathogenic (in other words, the repeat elements ‘AACCAT’ could cause the manifestation of disease in the patient. Thus, if any other near variations of the gene ‘X’ (i.e. other than variants ‘A’ and ‘B’), having a same stretch of the gene (e.g. AAAAATAAAAAT), it can be readily associated with the disease ‘D’ for any new patient. The variant associated with gene ‘X’ may be one of the learned variants of the method, system, medium or apparatus herein described.

Other examples of the variant may include but are not limited to, transcript ablation, splice donor variant, splice acceptor variant, stop gained, frameshift variant, start lost, initiator codon variant, transcript amplification, in-frame insertion, in-frame deletion, missense variant, protein-altering variant, splice region variant, incomplete terminal codon variant, synonymous variant, coding sequence variant, mature miRNA variant, 5 prime UTR variant, 3 prime UTR variant, non-coding transcript variant, intron variant, upstream variant, downstream variant, transcription factor (TF) binding site variant, regulatory region ablation, transcription factor binding sites (TFBS) ablation, and the like.

Learned variants or a collection thereof refers to variants that have been perceived or learned by a computational model. In other words, the collection of learned variants comprise variants or sequences of variants that the model has seen or considered as known or has been trained on by the model. Thus, a trained model with annotated variants or annotated data includes a data representation of learned variants underlying the interpretation information (that is quantified and for making decisions of pathogenicity based on patients and variants' annotations) of each variant, where the annotation is indicative of a particular observation(s) associated each variant for assessing whether the variant is phenotypically pathogenic (i.e. causing a given condition/disease) or benign (i.e. harmless) or the degree of pathogenic in the context of a set of pathogenic metrics. More specifically, the annotation provides the basis for assessing a likelihood of the variant being pathogenic given the model. The likelihood may be presented by probabilities or probability distributions in relation to the phenotypes exhibited.

The above-described computational model is thereby configured to assess any variant based on the set of pathogenic metrics, where pathogenic metrics is thereby trained by annotated variants that are known or thereafter as the collection of learned variants. Pathogenic metrics provide a classification scheme to which variants may be phenotypically categorized in relation to the degree of pathogenicity. Examples of these categories include but are not limited to, B (benign), LB (likely benign), LP (likely pathogenic), and P (pathogenic). Each of the categories are provided with the likelihood to which an indicative probability is determined. As such, the computational model can be a generative model configured to learn the data distribution of the training set so as to generate further data points or prediction with some variations with respect to the output probabilities.

The known variants or any variant sequences may be obtained from various data sources that include but are not limited to, for example, genomic databanks, public scientific databases, databases of research organizations (e.g. Database of Genomic Variants (DGV), Online Mendelian Inheritance in Man (OMIM), MORBID, DECIPHER, research literature (e.g. PubMed literature), and other supporting information, and so forth.

For example, in the case of OMIM, a gene name (e.g. ‘BICD2’ gene) and OMIM identifier (ID) (e.g. ‘609797’) are assigned to a variant. OMIM may include publicly available information on known mendelian disorders of about 15,000 genes, which is periodically updated and contain the relationship between phenotype and genotype. ‘MORBID ID’ (e.g. 615290) may also be assigned. A ‘MORBID ID’ is indicative of a chart or diagram of diseases and the chromosomal location of genes the diseases are associated therewith. The morbid map is provided in the OMIM knowledgebase, listing chromosomes and the genes mapped to specific sites on those chromosomes. Further, known conditions associated with the gene (e.g. the BICD2) gene may also be annotated (e.g. conditions: Proximal spinal muscular atrophy with autosomal-dominant inheritance). These annotations to the variant serve the basis for training the model.

In the training of the model, the annotated variants may be used for the derivation or generation of latent parameters coined herein as genetic condition clusters. These genetic condition clusters capture the abstract notion of the pathogenic categories to which an assessment of a gene of interest may be determined based on the pathogenic metrics. More specifically, the genetic condition clusters provide an abstract mapping to which a particular variant may relate to each of the phenotypic categories: B (benign), LB (likely benign), LP (likely pathogenic), and P (pathogenic) of the pathogenic metrics. In sum, the genetic condition clusters allow the prediction of a certain probability of pathogenicity for a given variant.

Various computational techniques may be used to derive these genetic condition clusters. These computation techniques may include one or more machine learning (ML) techniques, as herein described. These techniques may also include one or more matrix factorization algorithms that could be applied in collaborative filtering and recommender system applications where the aim is to model relational data by using latent parameters. Examples of these suitable methods include but are not limited to Latent Dirichlet Allocation, Non-Negative Matrix Factorization, Bayesian and non-Bayesian Probabilistic Matrix Factorization, Principal Component Analysis, Neural Network Matrix Factorization, and the like.

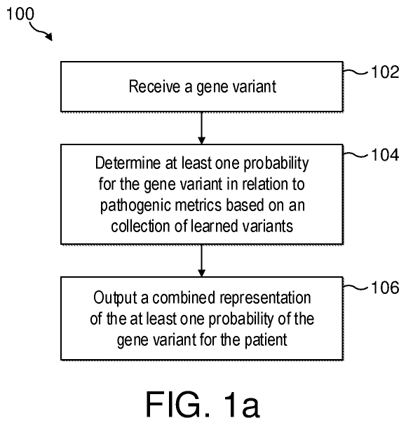

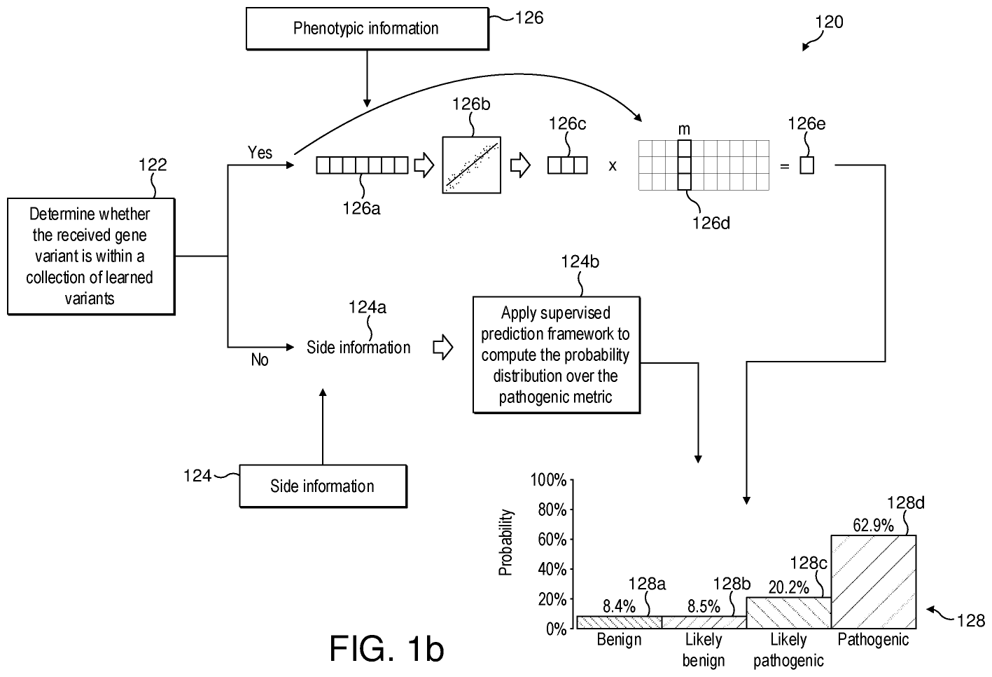

In applying the genetic condition clusters, evidence or a metric for a phenotypic category (i.e. benign) can be assessed to generate a probability associated with the particular category. The model may output a combined representation of each of the probability associated with the phenotypic categories for the interested variant for a patient. This combined representation may be in the form of a histogram, as shown in FIG. 1b or other graphical representation suitable for displaying the resultant probabilities of the model in combination.

Genetic condition clusters are weighted by a set of phenotypic information for fine-tuning the model by adjusting a certain contribution to the associated phenotype, while additional input of phenotypic information associated with a patient returns more accurate predictions based on the set of phenotypic information. In particular, the set of phenotypic information may be a matrix comprising phenotype data, for example Human Phenotype Ontology (HPO) terms or other coding of phenotype from available data sources, of a cohort of patients. The phenotype data are assigned, which provides a standardized way to represent phenotypic abnormalities encountered in human disease. In the case of HPO terms, they may be automatically retrieved if the gene sequence (e.g. BICD2) is previously reported as pathogenic and a part of the collection of learned variants. The HPO terms, for example, include “HP:0000347 ‘micrognathia’, HP:0001561 ‘polyhydramnios’, HP:0001989 ‘fetal akinesia sequence’, HP:0001790 ‘nonimmune hydrops fetalis’, HP:0002803 ‘congenital contracture. These HPO terms are used in combination with the genetic condition clusters during prediction based on the pathogenic metrics. More specifically, the HPO terms, or more generally phenotype data, are used to training weights associated with each of the genetic condition clusters. The training is accomplished using one or more ML techniques herein described or via curve fitting algorithms that include but are not limited to the use of linear regression with different penalty terms (i.e. LASSO, RIDGE, Elastic Net).

In addition to phenotypic information, a set of side information may be introduced to characterise the pathogenicity of unknown gene variants, that is, for variants that are not a part of the collection of learned variants. The set of side information or side information may refer to indicators associated with one or more gene variants herein described.

In particular, the set of side information pertains to one or more known variants learned by the model. Examples of side information include various phenotypic and genotypic indicators. These indicators include but are not limited to GERP score (defines the reduction in the number of substitutions in the multi-species sequence alignment compared to the neutral expectation), SIFT score (predicts whether an amino acid substitution affects protein function), Variant Effect Predictor (VEP) consequences (coordinates of the variant and the nucleotide changes associated with its effect), MVP score (predicts pathogenicity of missense variants via deep learning ML models). Alternatively, HI score and ADA score may also be used. For example, a HI score (e.g. 0.176) may be assigned to a variant of the gene with the indication of zygosity along with VEP consequence annotated for a known variant.

The prediction of the pathogenicity of unknown gene variants may be performed by using a supervised learning framework. Given an unknown gene variant and its side information, the prediction model(s) underlying the framework is configured to generate the probability for each pathogenic metrics (e.g., benign, likely benign, likely pathogenic, and pathogenic). That is, at least one model (M) computes the probability of the variant of being associated to each of these pathogenic metrics (Vm) given its side information (SI), or as M=P (Vm|SI).

The supervised learning framework or any of the underlying prediction model(s) may be trained by using the side information as independent variables and the pathogenic metrics (e.g., benign, likely benign, likely pathogenic, and pathogenic). The supervised learning framework may include a non-parametric classifier. The frameworks may also include but are not limited to linear regression, logistic regression, neural networks, Support Vector Machine (SVM), and the like. These models will generate different weights for the different side information that can be used to interpret the prediction (e.g., the GERP score can have a higher weight than the SIFT score, and this will result in GERP score having a more significant impact than SIFT score when computing the pathogenicity).

Machine learning (ML) techniques may be used to generate a trained model such as, without limitation, for example one or more generative ML models or classifiers based on input data referred to as training data associated with phenotypic and interpretation information. The input data may also include side information herein described. With correctly annotated training datasets in such fields as bioinformatics, techniques can be used to generate further trained ML models, classifiers, and/or generative models for use in downstream processes such as, by way of example but not limited to, drug discovery, identification, and optimization and other related biomedical products, treatment, analysis and/or modelling in the informatics, and/or bioinformatics fields.

Examples ML technique(s) for generating a trained model that may be used by the invention as described herein may include or be based on, by way of example only but is not limited to, one or more of: any ML technique or algorithm/method that can be used to generate a trained model; one or more supervised ML techniques; semi-supervised ML techniques; unsupervised ML techniques; linear and/or non-linear ML techniques; ML techniques associated with classification; ML techniques associated with regression and the like and/or combinations thereof. Some examples of ML techniques/model structures may include or be based on, by way of example only but is not limited to, one or more of active learning, multitask learning, transfer learning, neural message parsing, one-shot learning, dimensionality reduction, decision tree, association rule learning, similarity learning, data mining algorithms/methods, artificial neural networks (NNs), autoencoder/decoder structures, deep NNs, deep learning, deep learning ANNs, inductive logic programming, support vector machines (SVMs), sparse dictionary learning, clustering, Bayesian networks, reinforcement learning, representation learning, similarity and metric learning, sparse dictionary learning, genetic algorithms, rule-based machine learning, learning classifier systems, and/or one or more combinations thereof and the like.

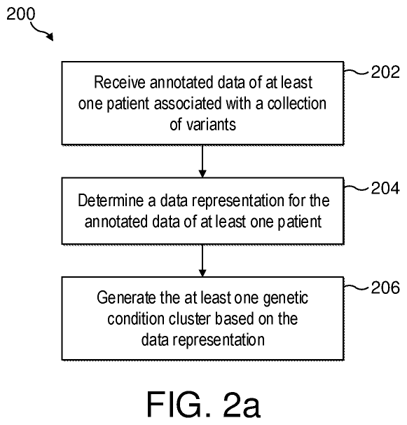

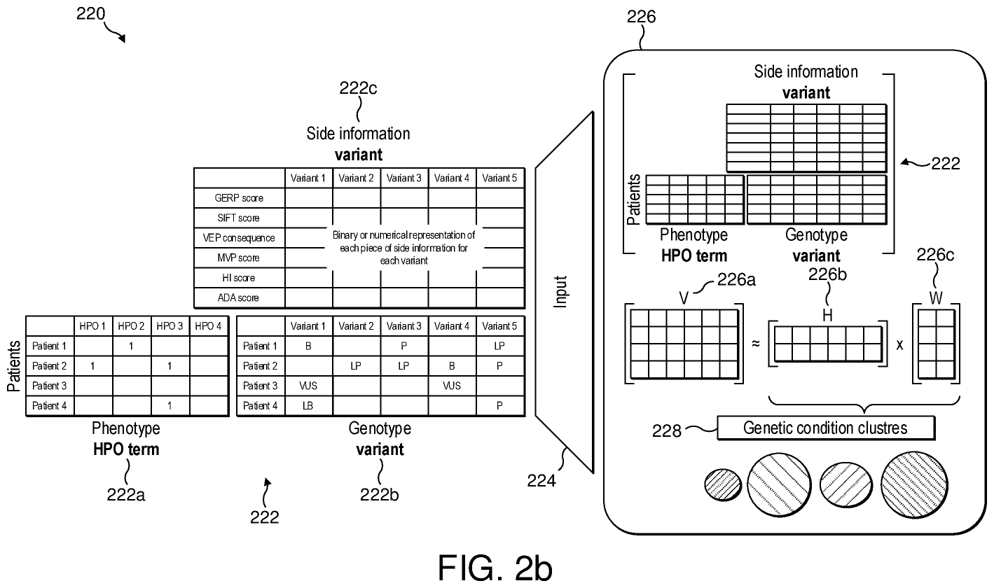

Types of training data or annotated data include but are not limited to the dataset associated with Patient ID, Patient Phenotype, Variant ID, Pathogenic Metric, and side information. Patient ID may be unique identifiers for each patient and shown as rows ID in matrices 222a and 222b of FIG. 2b. Patient Phenotype are phenotypes observed for the patients and may be presented as Human Phenotype Ontology (HPO) terms. One example of an HPO term is HP: 0000729 for patients with Autistic behaviour phenotype; and another example is HP: 000986 for patients with Limb undergrowth phenotype. HPO terms are shown as columns ID in the binary matrix 222a of FIG. 2b. Variant ID may be unique for each variant. Variant ID may present features that are concatenated and separated by underscore(s). For example, Variant ID 2_1765342_C_T_NM 00193456 uniquely identifies the variant on chromosome 2, starting at the base pair position 1765342, involving the mutation C>T on the transcript NM_00193456. Here, the Variant ID 2_1765342_C_T_NM_00193456 identifies the Chromosome, Start, Ref allele, Alt allele, and Transcript ID. Variant ID are shown as columns ID in the matrices 222b and 222c of FIG. 2b. Pathogenic Metric may be represented by the levels of the variant pathogenicity as designated by American College of Medical Genetics. For example, there may be a Pathogenic Metric B for Benign, LB for Likely Benign, LP for Likely Pathogenic, P for Pathogenic, and VUS for Uncertain Significance. These may be alternative training labels, for example, adapted to the matrix factorization algorithm and the entries shown in matrix 222b of FIG. 2b. The side information may be presented as variant's annotations used in the cosine similarity or organized in any suitable format used in a supervised learning framework. They are shown as columns ID of the matrix 222c of FIG. 2b.

The training data or annotated data are used for training the Pathogenicity Model to assess and compute the probability distribution for a gene variant in order to assess the pathogenicity of a variant for a patient. Specifically, the training data or annotated data may be organized in computer-readable formats that include but are not limited to a real number, binary, categorical, identifier, lists, and strings formats that are suitable for processing with one or more models, frameworks, algorithms, techniques, and methodologies here described.

A practical example of training data or annotated data in relation to the types of training data is shown in Table 1 below. The table also shows features associated with the side information for a given variant. For example, one feature may be the maximum allele frequency for the patient; another feature may be the non-synonymous amino acid change in a functional protein domain for the same patient. Each feature (of features 1 to 11) is presented in the table in relation to the Patient ID, Patient Phenotype, Variant ID, and Pathogenic Metric. The features may also correspond to the above described phenotypic and genotypic indicators that include but are not limited to GERP score, SIFT score, Variant Effect Predictor (VEP) consequences, MVP score. Other presentation of training data include the example in table 1 but are not limited to this example. Training data may be presented and organised in relation to the model, framework, algorithm, techniques, or methodology applied. The training data may be presented to accommodate as inputs for training the Pathogenicity Model as described herein.

TABLE 1

| Patient | Patient | | Pathogenic | Feature | Feature | Feature | | | | Feature |
|---|---|---|---|---|---|---|---|---|---|---|
| ID | Pheotypes | Variant ID | metric | 1 | 2 | 3 | Feature 4 | Feature 5 | Feature 6 | 7 |
| 1 | HP:000164 | 7_1506460 | B | 0 | 3.95 | | | frameshift_variant | | |
| 1 | HP:000164 | 11_768348 | LB | 0.005277 | −0.163 | | | missense_ | 0.002 | 0.64 |
| 1 | HP:000164 | 16_579939 | P | 0.000124 | −1.5 | 0.03 | 0.001013 | splice_region_variant | | |
| 2 | HP:000047 | 12_485164 | VUS | 0.218986 | 4.38 | 0.036 | 0.004091 | intron_variant | | |
| 3 | HP:000070 | 8_1007791 | B | 0.008287 | −2.49 | | | synonymous_variant | | |
| 3 | HP:000070 | 8_5553922 | LP | 0 | 4.2 | | | frameshift_variant | | |
| 3 | HP:000070 | 10_897208 | P | 0 | 4.39 | | | stop_gained | | |
| 4 | HP:000124 | 9_1194602 | B | 0 | 4.43 | 0.67 | 0.12 | synonymous_variant | | |
| 5 | HP:000047 | 3_3865144 | B | 0.006742 | 0.209 | 0.001 | 0.23 | synonymous_varianT | | |
| 5 | HP:000047 | 6_4268955 | P | 6.06E−05 | 5.78 | | | missense_ | 0.203 | 0.04 |
| 6 | HP:000048 | 5_8999044 | VUS | 0.003192 | 5.81 | | | missense_ | 0.018 | |
| 6 | HP:000048 | 5_7094598 | VUS | 0.00015 | 3.84 | 0.45 | 0.98 | missense_ | 0.037 | 0.05 |
| 7 | HP:000058 | 2_1795474 | LB | 0.01105 | −3.98 | | | synonymous_variant | | 0.352 |
| 7 | HP:000058 | 18_485934 | P | 1.00E−04 | 5.49 | 0.34 | 0.109 | missense_ | 0.912 | 0.04 |
| 8 | HP:000194 | 9_1171857 | VUS | 0.009235 | 4.41 | | | missense_ | 0.88 | |
| 8 | HP:000194 | 11_663347 | B | 0.000539 | −1 | 0.001 | 0.876 | synonymous_variant | | |
| 8 | HP:000194 | X_4907497 | LB | 0 | 4.73 | | | stop_gained | | |
| 9 | HP:000194 | 3_1506582 | VUS | 0.001079 | 0.649 | 0.762 | 0.999956 | splice_acceptor_variant | | |
| 9 | HP:000194 | 6_1372193 | LP | 0 | 5.96 | | | missense_ | 0.905 | 0.13 |
| 9 | HP:000194 | 10_735581 | B | 0.005642 | 4.63 | | | synonymous_variant | | |
| 9 | HP:000194 | 17_364935 | LP | 0.005394 | 3.1 | | | missense_ | 0.052 | 0.13 |
| 10 | HP:000194 | 10_735376 | B | 0.000458 | −11 | | | missense_variant | | |
| 11 | HP:000150 | 4_3634519 | LB | 0 | 2.58 | 0.987 | 0.567 | missense_ | 0.026 | 0.46 |

| 11 | HP:000150 | 15_784016 | P | 0.0032 | −7.53 | 0.26 | 0.02 | synonymous_variant | |
|---|---|---|---|---|---|---|---|---|---|
| 12 | HP:000047 | 11_119212 | VUS | 0.008287 | −6.19 | 0.4 | 0.6 | synonymous variant | |
| 13 | HP:000070 | 2_2024980 | B | 0.006272 | 1.46 | 0.6 | 0.24 | synonymous_variant | |
| | | | | | Patient | Feature | Feature | Feature | Feature |
| | | | | | ID | 8 | 9 | 10 | 11 |
| | | | | | 1 | 0.697 | | | 0 |
| | | | | | 1 | 0.208 | 5 | | 0 |
| | | | | | 1 | 0.68 | | | 1 |
| | | | | | 2 | 0.21 | | | 1 |
| | | | | | 3 | 0.277 | | Likely beni [symbol] | 0 |
| | | | | | 3 | 0.298 | | | 0 |
| | | | | | 3 | | | Pathogenic [symbol] | 0 |
| | | | | | 4 | 0.192 | | | 0 |
| | | | | | 5 | 0.242 | | Likely beni [symbol] | 0 |
| | | | | | 5 | 0.346 | 43 | | 0 |
| | | | | | 6 | 0.066 | 29 | Likely beni [symbol] | 0 |
| | | | | | 6 | 0.032 | 43 | | 0 |
| | | | | | 7 | 0.352 | | Likely beni [symbol] | 0 |
| | | | | | 7 | 1 | 32 | Uncertain [symbol] | 0 |
| | | | | | 8 | 0.248 | 98 | Likely beni [symbol] | 0 |
| | | | | | 8 | 0.109 | | | 0 |
| | | | | | 8 | 0.231 | | | 0 |
| | | | | | 9 | 0.166 | | Uncertain [symbol] | 1 |
| | | | | | 9 | 0.096 | 22 | | 0 |
| | | | | | 9 | 0.274 | | Likely beni [symbol] | 0 |
| | | | | | 9 | 0.07 | 43 | Uncertain [symbol] | 0 |
| | | | | | 10 | 0.274 | 23 | | 0 |
| | | | | | 11 | | 145 | | 0 |
| | | | | | 11 | 0.313 | | | 0 |
| | | | | | 12 | 0.158 | | Likely beni [symbol] | 0 |
| | | | | | 13 | 0.073 | | Likely beni [symbol] | 0 |

[symbol]

indicates data missing or illegible when filed

FIG. 1a is a flow diagram illustrating an example process 100 of assessing pathogenicity of a variant for a patient according to the invention. The level of pathogenicity may be assessed by at least one predictive model that is trained using annotated data. The steps of assessing pathogenicity of a variant by process 100 are as follows:

In step 102, a variant is received associated with the patient. The variant may be either a variant known to the model or a variant that is unknown. Additionally or alternatively, together with the variant, phenotypic information of the patient may also be used for the assessment of the pathogenicity.

In step 104, at least one probability for the variant is determined in relation to the pathogenic metrics of the predictive model. The predictive model is trained to retain data representation of a collection of variants or variant learned by the model. The collection of learned variants comprises a data representation of at least one genetic condition cluster in making the determination of the at least one probability for the variant as such. Additionally or alternatively, a data representation of the at least one genetic condition cluster is derived from the collection of learned variant and weighted in relation to the set of phenotypic information of patients. The availability of phenotypic information of the patient assessed and determined to the extent in the absence of the phenotypic information of the patient, adjustment to the at least one genetic condition cluster for outputting the combined representation may be considered. As an option, the combined representation, probabilities generated for each of phenotypic metrics, may be normalised to 100% or 1 in relation to the respective probabilities.

In step 106, at least one probability of the variant for the patient is outputted. The output may be a combined representation of the probabilities generated. In one example, the output may be part of an interface where the user may consider the underlying probabilities as having an automated assistant preparing user's interpretation for review. More specifically, together with the combined representation of the probabilities, the interface may prompt at least one output that includes but are not limited specified labels corresponding the level of pathogenicity, contribution to phenotype, report category and the like. Further explanatory information may be presented as part of the combined output.

Additionally or alternatively, once the phenotypic information of the patient is received provided that the variant is included in the collection of learned variants to the extent that the variant is considered known to the at least one predictive model, the contribution associated with each of the at least one genetic condition cluster based on the phenotypic information of the patient can be determined. With this determination, as an option, each of the at least one genetic condition cluster is portioned using one or more regression models of the at least one predictive models. The one or more regression models predict the contribution to each of the at least one genetic condition cluster given the phenotypic information of the patient. In accordance, the at least one probability for the variants is adjusted based on the contribution in relation to the data representation of the at least one genetic condition cluster. In effect, the contribution provides improved accuracy with aligned with the phenotypic information provided.

In the case where an unknown variant presented to the at least one predictive model such that the variant is not included in the collection of learned variants, a supervised learning framework is used to compute the probability distribution over the pathogenic metrics given the set of side information of the unknown variant, which may comprise one or more phenotypic and/or genomic indicators. In effect, any variant unknown or unseen to the predictive model may be assessed accordingly based on the reservoir or collection of known or learned variants.

FIG. 1b is a schematic diagram illustrating an example process 120 where the pathogenicity of a variant for a patient is assessed in relation to phenotypic 126 and side information 124 according to the invention based on the example process 100 described with reference to FIG. 1a. A determination 122 of whether the received variant is within the collection of learned variants is made. If “yes” then the variant received is known to the predictive model, the phenotypic information of the patient is applied in determining the contribution to the latent variables or genetic condition clusters. The genetic condition clusters as derived by one or more generative models or ML models, or applying ML techniques herein described, in turn, provides an empirical evaluation for the pathogenicity based on pathogenic metrics.

In one example, the patient's HPO terms 126a may be used in accordance with a linear regression model 126b to determine the degree of contribution 126c for each of the latent variables. The latent variables are derived using LDA, where matrix decomposition is performed. In accordance, the evidence or probability of whether the inputted variant is benign or another pathogenic metrics may be determined using either additional phenotypic information of the patient and/or with the received variant directly by applying the latent variables or hidden genetic condition clusters. Similarity probabilities may be determined based on the pathogenic metrics such as, for example, benign, likely benign, likely pathogenic, and pathogenic. That is, pathogenic metrics may comprise at least one classification indicative of a degree or level of pathogenic. The at least one classification may be associated with a different optimal set of the at least one genetic condition cluster such that a combined representation 128 of these metrics with underlying probabilities for benign 128a, likely benign 128b, likely pathogenic 128c, and pathogenic 128d may be presented and outputted.

In the case of “no” then the variant received is unknown to the predictive model, further side information 124 attributing the one or more phenotypic and/or genomic indicators may be used in relation to a supervised learning framework. The supervised learning framework may be applied to compute the probability distribution the pathogenic metric 124b based on received side information 124a. The side information serves to evaluate the resultant probabilities, indicative of a degree of pathogenic, associated with the pathogenic metrics. In effect, the application of side information overcomes the dilemma where an unknown variant is presented to the predictive model.

FIG. 2a is a flow diagram illustrating an example process 200 of generating genetic condition clusters for determining at least one probability of a variant in relation to pathogenic metrics according to the invention. In this example, annotated data is used to train the predictive model. Specifically, annotated data is used to derive the hidden genetic condition clusters associated with at least one generative model or ML model, or applying one or more ML techniques herein described. In this example, the process 200 of generating genetic clusters may include the following steps of:

In step 202, the annotated data of at least one patient associated with a collection of variants is received. The received annotated data may comprise interpretation information and observations corresponding to the pathogenic metric. The interpretation information may be genotypic in nature. Additionally or alternatively, the annotated data may further comprise a set of phenotypic information of patients, that is associated with the interpretation information in relation to the at least one patient and/or a set of side information, that is associated with the interpretation information in relation to the collection of variants to the extent that the set of side information may include a data representation of indicators associated with the collection of variants.

In particular, the set of side information may be used, when the variant is not included in the collection of variants or not received as part of the annotated data, to compute the probability distribution over the pathogenic metrics by using a supervised learning framework.

As an option, a set of weights associated with at least one genetic condition cluster may be adjusted based on the set of phenotypic information. The set of weight may correspond to a contribution of the at least one genetic condition cluster to the set of phenotypic information. One or more regression models may be configured based on the adjusted set of weights to determine the contribution in relation to the pathogenic metrics. One or more ML models or techniques may also be applied alternatively or additionally to attain the contribution to the genetic condition clusters.

In step 204, a data representation for the received annotated data of at least one patient may be determined and derived using one or more generative models or corresponding ML models, or ML techniques herein described. The one or more generative models are configured to decompose the data presentation of annotated data in relation to the pathogenic metrics. For example, a matrix factorization algorithm such as and LDA may be applied.

In this example, the hidden genetic condition clusters of the LDA are abstract parameters that are derived using the decomposition of the multi-dimensional data matrix of patients, variants and corresponding observations. The derived genetic condition cluster enables a compilation of probabilities that may be used to assess pathogenicity for a given variant. Following the decomposition or factorization of the multi-dimensional data matrix, the optimal number of genetic condition clusters may be determined, for example, by using Expectation-Maximization. As such, the number of genetic condition clusters may change as the predictive model increments with more data. Alterative techniques such k-fold cross-validation (e.g. k=5) may also be applicable in that the optimal number of genetic condition clusters can be determined and scored using the notion of perplexity as evaluation score—the optimal solution is the one minimizing the perplexity. The different decomposition, in this case, should be performed for each binary matrix associated with a phenotypic metric such that each decomposition may have a different optimal number of genetic condition clusters or latent variables.

In step 206, at least one genetic condition cluster is generated based on the data representation. The data representation may be abstract parameters or alternatively ML features of one or more ML models as described herein. The one or more ML models or techniques may also be used to determine an optimal set of the at least one genetic condition cluster based on the annotated data in addition to or in conjunction with the techniques described in any of the examples of this application. In turn, the optimal set of at least one genetic condition cluster could be used to predict at least one probability of a variant in relation to the pathogenic metrics. Additionally or alternatively, the optimal set of the at least one genetic condition cluster may be configured to be updated iteratively with new or additional annotated data.

FIG. 2b is a schematic diagram of an example process 220 of genetic condition clusters for determining a probability of a variant according to the invention based on the example process 200 described with reference to FIG. 2a. In order to generate the genetic condition clusters 228, a data representation of a multi-dimensional data matrix 222 may serve as input 224 for the determination of the clusters. In particular, the data matrix 222 incorporates information of the patients, variants and corresponding observations (“labelled data” from past patient interpretations). It is often the case that observations in the matrix are highly sparse relative to the size of the matrix, —99.96% of the observation ‘cells’ are empty because there are so many variants possible.

More specifically, the multi-dimensional data matrix 222 may be presented in terms of phenotype information matrix 222a, interpretation information matrix 222b, and side information matrix 222c with respect to data associated with patients, variants and corresponding observations. In particular, the interpretation information matrix 222b may be decomposed to generate the genetic condition clusters. An example of the phenotype information may include HPO terms (HPOs 1 to 3 present in patient 1 to 4), and interpretation information may include variants or a collection thereof (where, for example, patient1 has two variants labelled as pathogenic, and patient3 has no pathogenic variants). The side information matrix, on the other hand, corresponds to phenotypic and genotypic indicators such as GREP score, SIFT score, VEP consequences, MVP score, HI score, ADA score and the like. The side information matrix 222c, for example, may comprise columns that contain real numbers (i.e., max allele frequency), and columns containing categorical variables (i.e., VEP consequence). The categorical variables may be transformed into an integer (binary) representation by using a dummy coding scheme. Thus, each patient has side information (or binary vector) describing the patient's phenotypes (or signs/symptoms) as HPO terms or applying other phenotype coding schemas (e.g. OMIM, IDC10, and the like). The matrix that contains the HPOs or the quantitative value thereof for all patients in the data set may be used to train, for example, a regression model, for the determination of the genetic condition clusters.

Further in FIG. 2b, the interpretation information matrix in relation to the pathogenicity metrics (e.g. B, LB, P, LP) is decomposed (i.e. broken down into H 226b and W 226c, which multiply back together to get V 226a). The decomposition of the interpretation information matrix generates a number of binary matrixes equal to the number of pathogenicity metrics. Here, the matrix W 226c is used to represent the proportion of each genetic condition cluster 228 inside each patient in the training data set. The matrix H 226b contains the number of times each variant is associated with each genetic condition cluster 228. Therefore, the genetic condition clusters are simply one dimension of the matrix decomposition. In turn, matrix factorization algorithms such as LDA via Expectation-Maximization may be applied to optimize a finite set of genetic condition clusters. The finite set of the genetic condition clusters may be determined by the use of validation techniques (e.g. k-fold). The optimal numbers (e.g. 5, 6, 7 . . . 25) of the finite set of genetic conditions clusters 228 may be stored and continue to be updated as different numbers of genetic condition clusters become or determined to be optimal during the validation techniques. In effect, given the four decompositions corresponding to the four pathogenic levels, predictions for any variant contained in the collection of the learned variant may be determined.

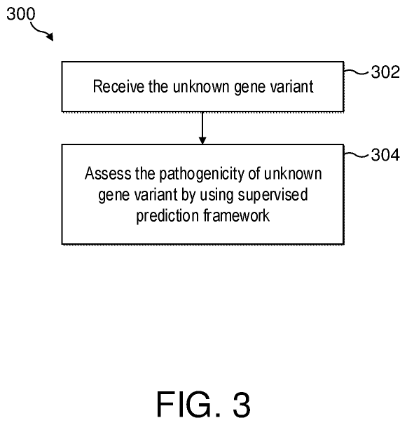

FIG. 3 is a flow diagram illustrating an example process 300 of assessing pathogenicity of an unknown variant for a patient using a set of side information according to the invention. Any unknown variant is a variant that is not included in the collection of learned variants to which the predictive model has learned. Based on the side information of the unknown variant, the probability distribution over the pathogenic metrics by using a supervised prediction model.

In step 302, an unknown variant, which is not identified in the collection of learned variants, is received. The received unknown variant could be any variant of the patient that has not been seen by the predictive model or specifically classified by genetic condition clusters.

In step 304, the pathogenicity of the unknown variant may be assessed. This assessment is made by using a supervised learning framework, which includes one or more supervised prediction models, which generates a probability for each pathogenic metric given the variant's side information. For example, the output may be in the form of a histogram displaying the normalized probabilities for each metric.

As a different option, a set of side information corresponding to each of a subset of the collection of learned variants is compared to determine the nearest variant. As another option, the set of side information corresponding to each of the subsets of the collection of learned variants is compared in relation to similarity scores. For example, the similarity scores may be cosine similarity scores or other suitable scoring methods that are adapted to assess the subset of the collection of learned variants to determine the nearest variant.

As another option, pathogenicity of the unknown variant, in relation to the pathogenicity of the nearest variant, may be assessed. In particular, at least one probability for the nearest variant based on a collection of learned variants may be determined. This determination is made in relation to the pathogenic metrics that comprise a data representation of at least one genetic condition cluster. That is, the at last one genetic condition cluster may be applied to compute the at least one probability for the nearest variant. At least one probability computed may be complied to introduce a combined representation, where the combined representation is outputted with respect to the pathogenic metrics. The output may, for example, in the form a histogram displaying the normalized probabilities for each metrics. Additionally or alternatively, the combined representation may be generated by averaging the at least one probability for each variant of a subset of the collection of learned variants, in response to the subset of the collection of learned variants comprise two or more variants with equivalent similarity score such that the nearest variant cannot be determined.

As another option, the pathogenic metrics of any of the examples described herein may comprise at least one classification indicative of a degree of pathogenic. Each of the at least one classification may be further associated with a different optimal set of the at least one genetic condition cluster. The optimal set of genetic condition may be determined when applying, for example, LDA in conjunction with Expectation-Maximization or alternatively via one or more ML models or techniques described herein. Specifically, suitable validation techniques may also be applicable for determining the number of genetic condition clusters in the optimal set, for example by minimizing perplexity, such that each decomposition could have a different optimal number of genetic condition clusters. The different optimal number of genetic conditions may be derived, for each binary matrix associated with a phenotypic metric, by using any technique for determining the optimal number of genetic condition clusters described herein.

As another option, weighted similarity metrics may be used to identify or determine a best nearest variant or variant that is most similar to the unknown variant with respect to the weighted similarity metrics. The weighted similarity metrics may retain different or similar weights for different side information. Specifically, one score of the side information may have a higher weight than another score, and the higher score will have a greater impact when computing the nearest variant. The aim of using weighted similarity metrics is to take into account the predictive power specific of each side information and enhance the process of identification of the best nearest learned variant. These weights can be inferred by using both linear and non-linear models associated with the one or more ML techniques herein described.

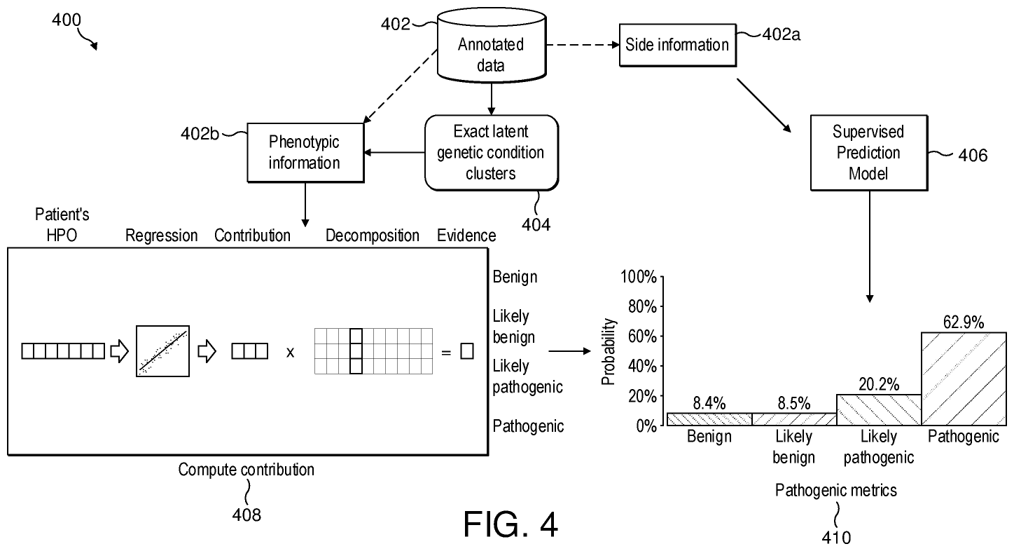

FIG. 4 is a schematic diagram illustrating an example process 400 of genetic condition clusters extracted from annotated data to predict probabilities of the variant given the pathogenic metrics according to the invention with reference to FIGS. 1a to 3. In the example, the latent or hidden genetic clusters or latent variables underlying the predictive model may be extracted from the annotated data, which is used as the training data set for the model. The data set may be in the form of a multi-dimensional data matrix comprises data points associated with patients, variants, and corresponding observations numerically presented in the matrix. The extracted genetic condition clusters may be a single dimension (vector) of the matrix generated upon the decomposition procedure. Each decomposition is associated with a pathogenic metric (B, LP, P, and LP) as shown in the figure. Alterative pathogenic metrics with varying degree of pathogenicity, other than the shown metrics, may also be applicable. With four decompositions deduced, predictions of pathogenicity can be made for any variant that resides in the annotated data. In the figure, the decomposition is achieved by performing LDA on the matrix with resultant decompositions for each of the pathogenic metric. The decomposition procedure may be accomplished alternatively using a number of other techniques, which include one or more ML techniques described with the aim to reduce the dimensionality of the data. The resultant vector of genetic condition clusters, therefore effectively embodies the annotated data.

Further, in this example, the genetic condition clusters may be weighted in relation to phenotypic information 402b. The weighting of the genetic condition clusters resolves the situation where the predictions turn out to be the same for patients having different phenotypes. The accuracy of the predictive model, therefore, increases due to the fact patients' phenotypes may be included as part of the model's framework, and resultant predictions may be linked to the specific characteristics of each patient. As shown in the figure, a linear regression model as an example is used with the aim to predict or compute the contribution 408 of each genetic condition cluster given phenotypic information such as the HPO terms of a patient. These examples of HPO terms may be used to adjust the overall probability of the generated profile by associating a weight to each genetic condition cluster. As an option, where no HPO terms are provided as input, then there no weighting is applied to the genetic condition clusters. The profile generated for each patient and a particular variant may be shown as the normalised probabilities based on pathogenicity metrics 410.

Alternatively or additionally, side information 402a may be used where the input variant of the patient is not present in the annotated data or a part of learned variants associated with the genetic condition cluster. In other words, when a new or unknown variant is presented to the predictive model, a supervised prediction model 406 may use the side information 402a to determine the probability distribution over the pathogenic metric for the unknown variant without having to retrain the predictive model on a known interpretation.

As an example, a supervised learning framework may be used to compute the pathogenicity by using the side information 402a described herein. Thus, the predictive model is above to predict both known and unknown variants without retrained for the required accuracy upon meeting an unknown variant and enhancing model sustainability.

As a different option, side information may be used where the input variant of the patient is not present in the annotated data or a part of learned variants associated with the genetic condition cluster. In other words, when a new or unknown variant is presented to the predictive model, using side information to determine the nearest variant without having to retrain the predictive model on a known interpretation (and generating/updating new genetic condition clusters).

In the different option, cosine similarity may be used to plot the variants on a multi-dimensional chart. Using one or more of the side information as described herein, the nearest or variant with the small distance (based on the cosine similarity score) to the collection of the learned variant may be determined as the predicted variant. In particular, the variant having the most similar cosine score or effectively with similar variant side information is identified from the multi-dimensional chart. The predicted variant would replace the inputted variant for the purpose of generating the profile for each patient and the inputted variant. That is, the entry of the nearest neighbour in the matrix H is then used as a proxy for the unknown variant and generate a probability prediction in the same way as if the variant was known. If two or more variants have the same (argmax) cosine similarity score, then the final probability is computed by averaging the results across all selected variants. Thus, the predictive model is above to predict both known and unknown variants without having to be retrained for the required accuracy upon meeting an unknown variant and enhances model sustainability.

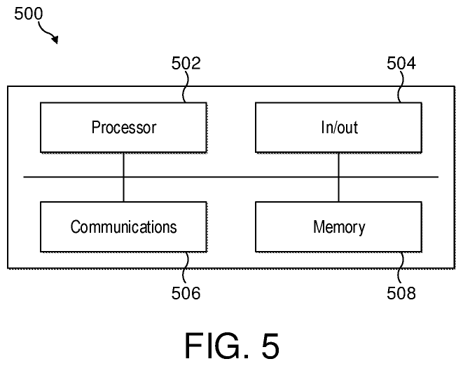

FIG. 5 is a schematic diagram illustrating an example computing apparatus/system 500 that may be used to implement one or more aspects of the predictive model, apparatus, method(s), and/or process(es) combinations thereof, modifications thereof, and/or as described with reference to FIGS. 1a to 4 and/or as described herein. Computing apparatus/system 500 includes one or more processor unit(s) 502, an input/output unit 504, communications unit/interface 506, a memory unit 508 in which the one or more processor unit(s) 502 are connected to the input/output unit 504, communications unit/interface 506, and the memory unit 508. In some embodiments, the computing apparatus/system 500 may be a server, or one or more servers networked together. In some embodiments, the computing apparatus/system 500 may be a computer or supercomputer/processing facility or hardware/software suitable for processing or performing the one or more aspects of the predictive model for pathogenicity assessment system(s), apparatus, method(s), and/or process(es) combinations thereof, modifications thereof, and/or as described with reference to FIGS. 1a to 4 and/or as described herein. The communications interface 506 may connect the computing apparatus/system 500, via a communication network, with one or more services, devices, server system(s), cloud-based platforms, systems for implementing subject-matter databases and/or knowledge graphs for implementing the invention as described herein. The memory unit 508 may store one or more program instructions, code or components such as, by way of example only but not limited to, an operating system and/or code/component(s) associated with the assessment of variants process(es)/method(s) as described with reference to FIGS. 1a to 4, additional data, applications, application firmware/software and/or further program instructions, code and/or components associated with implementing the functionality and/or one or more function(s) or functionality associated with one or more of the method(s) and/or process(es) of the device, service and/or server(s) hosting the predictive model for pathogenicity assessment process(es)/method(s)/system(s), apparatus, mechanisms and/or system(s)/platforms/architectures for implementing the invention as described herein, combinations thereof, modifications thereof, and/or as described with reference to at least one of figure(s) 1a to 4.

In the embodiments, examples, of the invention as described above such as the predictive model for pathogenicity assessment process(es), method(s), system(s) and/or apparatus may be implemented on and/or comprise one or more cloud platforms, one or more server(s) or computing system(s) or device(s). A server may comprise a single server or network of servers, the cloud platform may include a plurality of servers or network of servers. In some examples the functionality of the server and/or cloud platform may be provided by a network of servers distributed across a geographical area, such as a worldwide distributed network of servers, and a user may be connected to an appropriate one of the network of servers based upon a user location and the like.

In an aspect associated with FIGS. 1a to 4, a computer-implemented method for assessing pathogenicity of a variant for a patient comprising: receiving a variant; determining at least one probability for the variant in relation to pathogenic metrics based on a collection of learned variants, wherein the pathogenic metrics comprise a data representation of at least one genetic condition cluster for determining the at least one probability for the variant; and outputting a combined representation of the at least one probability of the variant for the patient.

In another aspect, a computer-implemented method for generating at least one genetic condition cluster for determining at least one probability of a variant in relation to pathogenic metrics comprising: receiving annotated data of at least one patient associated with a collection of variants, wherein the annotated data comprise interpretation information with associated observations corresponding to the pathogenic metrics; determining a data representation for the annotated data of at least one patient, wherein the data representation is derived using one or more generative models; and generating the at least one genetic condition cluster based on the data representation.

In yet another aspect, a computer-implemented method for assessing pathogenicity of an unknown variant for a patient using a set of side information comprising: receiving the unknown variant, wherein the unknown variant is not identified in the collection of learned variants; using the set of side information corresponding to each of a subset of the collection of learned variants to train a supervised learning framework; and assessing the pathogenicity of the unknown variant based on the supervised learning framework.

In yet another aspect, a computer-readable medium comprising computer-readable code or instructions stored thereon, which when executed on a processor, causes the processor to implement the computer-implemented method according to any steps optionally described below.

In yet another aspect, a system comprising at least one circuitry that is configured execute the computer-implemented method according to any steps optionally described below.

In yet another aspect, an apparatus comprising a processor, a memory and a communication interface, the processor connected to the memory and communication interface, wherein the apparatus is adapted or configured to implement the steps according to any optionally described below.

In yet another aspect, an apparatus for determining pathogenicity of a variant for a patient, the apparatus comprising: an input component configured to receive the variant; a processing component configured to determine whether the variant is within a collection of learned variants; a prediction component, in response to a determination that the variant is present in the collection of the learned variant, configured to generate at least one probability for the variant in relation to pathogenic metrics, wherein the pathogenic metrics comprise a data representation of at least one genetic condition cluster for determining the at least one probability for the variant; and a display component configured to display the at least one probability for the variant with respect to the pathogenic metrics, wherein the at least one probability is normalised.

In yet another aspect is a computer-implemented method for determining a probability distribution of pathogenicity for an unknown gene variant using a set of side information, the method comprising: receiving the unknown variant of a patient, wherein the unknown variant is not identified in or new to the collection of learned variants associated with a plurality of patients; assessing the pathogenicity of the unknown gene variant by using a supervised learning framework based on the set of side information; and determining the probability distribution of pathogenicity based on the assessment.

The following optional steps pertains to any one or more of the above aspects where appropriate.

Optionally, the prediction component, in response to a determination that the variant is absent in the collection of the learned variant, configured to receive a set of side information, wherein the side information is used to identify, in relation to the variant, a nearest variant that is applied as the variant to generate the at least one probability.

Optionally, the input component configured to receive phenotypic information associated with the patient, wherein the phenotypic information is applied to adjust the at least one probability for the variant in relation to the at least one genetic condition cluster.

Optionally, the data representation of the at least one genetic condition cluster is derived from the collection of learned variant and weighted in relation to a set of phenotypic information of patients.

Optionally, the variant is included in the collection of learned variants, further comprising: receiving phenotypic information of the patient; determining a contribution associated with each of the at least one genetic condition cluster based on the phenotypic information of the patient; and adjusting the at least one probability for the variants based on the contribution determined in accordance with the data representation of the at least one genetic condition cluster.

Optionally, the computer-implemented method further comprising: assessing an availability of the phenotypic information of the patient; and determining, based on the availability, whether to adjust the at least one genetic condition cluster for outputting the combined representation.

Optionally, the determining a contribution associated with each of the at least one genetic condition cluster based on the phenotypic information of the patient, further comprising: portioning each of the at least one genetic condition cluster using one or more regression models, wherein the one or more regression models predict the contribution to each of the at least one genetic condition cluster given the phenotypic information of the patient.

Optionally, the variant is not included in the collection of learned variants, further comprising: identifying at least one proximal variant from the collection of learned variants in relation to the variant; receiving a set of side information corresponding to each of the at least one proximal variant, wherein the set of side information comprises one or more indicators; identifying a nearest variant based on the set of side information; and applying the nearest variant as the variant when determining the at least one probability for the variant in relation to the pathogenic metrics.

Optionally, the nearest variant is identified by applying similarity metrics associated with the at least one proximal variant based on the set of side information.

Optionally, the similarity metrics are weighted in relation to the set of side information

Optionally, when the similarity metrics identify at least one other variant from the collection of learned variants to have an equivalent similarity score, the at least one probability for the variant is determined by averaging each of the at least one proximal variant.

Optionally, the annotated data further comprises a set of phenotypic information of patients and/or a set of side information.

Optionally, the set of phenotypic information is associated with the interpretation information in relation to the at least one patient; and/or wherein the set of side information is associated with the interpretation information in relation to the collection of variants.

Optionally, the computer-implemented method further comprising: adjusting a set of weights associated with the at least one genetic condition cluster based on the set of phenotypic information, wherein the set of weight corresponds to a contribution of the at least one genetic condition cluster to the set of phenotypic information; and configuring one or more regression models based on the adjusted set of weights to determine the contribution in relation to the pathogenic metrics.

Optionally, the set of side information comprises a data representation of indicators associated with the collection of variants.

Optionally, the set of side information is applied, when the variant is not included in the collection of variants, to identify a nearest variant from the collection of variants used for determining the at least one probability of the variant.

Optionally, the variant is included in the collection of variants for updating the least one genetic condition cluster by applying annotation associated with the nearest variant.

Optionally, the computer-implemented method further comprising: determining an optimal set of the at least one genetic condition cluster based on the annotated data; and applying the optimal set of the at least one genetic condition cluster during prediction to determine the at least one probability of a variant in relation to the pathogenic metrics.

Optionally, the optimal set of the at least one genetic condition cluster is configured to be updated iteratively with new annotated data.

Optionally, the set of side information corresponding to each subsets of the collection of learned variants is compared in relation to similarity scores associated with the subsets of the collection of learned variants.

Optionally, the assessing the pathogenicity of the unknown variant in relation to the pathogenicity of the nearest variant further comprising: determining at least one probability for the nearest variant in relation to pathogenic metrics based on a collection of learned variants, wherein the pathogenic metrics comprise a data representation of at least one genetic condition cluster for computing the at least one probability for the nearest variant; and generating a combined representation of the at least one probability, wherein the combined representation is outputted with respect to the pathogenic metrics.

Optionally, the computer-implemented method further comprising: generating the combined representation by averaging the at least one probability for each variant of a subset of the collection of learned variants, in response to the subset of the collection of learned variants comprise two or more variants with equivalent similarity score such that the nearest variant cannot be determined.

Optionally, the phenotypic information comprises phenotypic ontology associated with one or more diseases.

Optionally, the one or more generative models are configured to decompose the data presentation of annotated data in relation to the pathogenic metrics.

Optionally, the one or more generative models comprise at least one formulation based on a matrix factorization algorithm.

Optionally, the pathogenic metrics comprises at least one classification indicative of a degree of pathogenic.

Optionally, the each of the at least one classification is associated with a different optimal set of the at least one genetic condition cluster.

Optionally, further computing a probability of the unknown variant associated with a set of pathogenic metrics given the set of side information.

Optionally, further determining at least one probability for the unknown variant in relation to pathogenic metrics based on a collection of learned variants; and generating a combined representation of the at least one probability, wherein the combined representation is outputted with respect to the pathogenic metrics.

Optionally, the pathogenic metrics comprise a data representation of at least one genetic condition cluster for computing the at least one probability for a nearest variant.

Optionally, the supervised learning framework comprises one or more prediction models.

Optionally, the supervised learning framework comprises a non-parametric classifier.

Optionally, the set of side information is associated with the unknown gene variant.

The above description discusses embodiments of the invention with reference to a single user for clarity. It will be understood that in practice the system may be shared by a plurality of users, and possibly by a very large number of users simultaneously.

The embodiments described above may be configured to be semi-automatic and/or are configured to be fully automatic. In some examples a user or operator of the predictive model for pathogenicity assessment system(s)/process(es)/method(s) may manually instruct some steps of the process(es)/method(es) to be carried out.

The described embodiments of the invention the predictive model for pathogenicity assessment system, process(es), method(s) and/or apparatus and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, IoT devices, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method for assessing pathogenicity of a variant for a patient comprising:
receiving a variant;
determining at least one probability for the variant in relation to pathogenic metrics based on a collection of learned variants, wherein the pathogenic metrics comprise a data representation of at least one genetic condition cluster for determining the at least one probability for the variant; and
outputting a combined representation of the at least one probability of the variant for the patient.

2. The computer-implemented method of claim 1, wherein the data representation of the at least one genetic condition cluster is derived from the collection of learned variants and weighted in relation to a set of phenotypic information of patients.

3. The computer-implemented method of claim 1, wherein the variant is included in the collection of learned variants, further comprising:
receiving phenotypic information of the patient;
determining a contribution associated with each of the at least one genetic condition cluster based on the phenotypic information of the patient; and
adjusting the at least one probability for the variant based on the contribution determined in accordance with the data representation of the at least one genetic condition cluster.

4. The computer-implemented method of claim 2, further comprising:
assessing an availability of the phenotypic information of the patient; and
determining, based on the availability, whether to adjust the at least one genetic condition cluster for outputting the combined representation.

5. The computer-implemented method of claim 3, wherein the determining a contribution associated with each of the at least one genetic condition cluster based on the phenotypic information of the patient, further comprising:
portioning each of the at least one genetic condition cluster using one or more regression models, wherein the one or more regression models predict the contribution to each of the at least one genetic condition cluster given the phenotypic information of the patient.

6. The computer-implemented method of claim 1, wherein the variant is not included in the collection of learned variants, further comprising:
identifying at least one proximal variant from the collection of learned variants in relation to the variant;
receiving a set of side information corresponding to each of the at least one proximal variant, wherein the set of side information comprises one or more indicators;
identifying a nearest variant based on the set of side information; and applying the nearest variant as the variant when determining the at least one probability for the variant in relation to the pathogenic metrics.

7. The computer-implemented method of claim 6, wherein the nearest variant is identified by applying similarity metrics associated with the at least one proximal variant based on the set of side information; and/or wherein the similarity metrics are weighted in relation to the set of side information.

8. The computer-implemented method of claim 7, when the similarity metrics identify at least one other variant from the collection of learned variants to have an equivalent similarity score, the at least one probability for the variant is determined by averaging each of the at least one proximal variant.

9. A computer-implemented method for generating at least one genetic condition cluster for determining at least one probability of a variant in relation to pathogenic metrics comprising:
receiving annotated data of at least one patient associated with a collection of variants, wherein the annotated data comprise interpretation information with associated observations corresponding to the pathogenic metrics;
determining a data representation for the annotated data of at least one patient, wherein the data representation is derived using one or more generative models; and generating the at least one genetic condition cluster based on the data representation.

10. The computer-implemented method of claim 9, wherein the annotated data further comprises at least one of a set of phenotypic information of patients and a set of side information.

11. The computer implemented method of claim 10, wherein at least one of
the set of phenotypic information is associated with the interpretation information in relation to the at least one patient; and
wherein the set of side information is associated with the interpretation information in relation to the collection of variants.

12. The computer-implemented method of claim 10, further comprising:
adjusting a set of weights associated with the at least one genetic condition cluster based on the set of phenotypic information, wherein the set of weights corresponds to a contribution of the at least one genetic condition cluster to the set of phenotypic information; and
configuring one or more regression models based on the adjusted set of weights to determine the contribution in relation to the pathogenic metrics.

13. The computer-implemented method of claim 10, wherein the set of side information comprises a data representation of indicators associated with the collection of variants.

14. The computer-implemented method of claim 10, wherein the set of side information is applied, when the variant is not included in the collection of variants, to identify a nearest variant from the collection of variants used for determining the at least one probability of the variant; and/or wherein the at least one probability of the variant is determined using a supervised learning framework provided the set of side information.

15. The computer-implemented method of claim 14, wherein the variant is included in the collection of variants for updating the least one genetic condition cluster by applying annotation associated with the nearest variant.

16. The computer-implemented method of claim 9, further comprising:
determining an optimal set of the at least one genetic condition cluster based on the annotated data; and
applying the optimal set of the at least one genetic condition cluster during prediction to determine the at least one probability of a variant in relation to the pathogenic metrics.

17. The computer-implemented method of claim 16, wherein the optimal set of the at least one genetic condition cluster is configured to be updated iteratively with new annotated data.

18. A computer-implemented method for assessing pathogenicity of an unknown variant for a patient using a set of side information comprising:
receiving the unknown variant, wherein the unknown variant is not identified in the collection of learned variants;
using the set of side information corresponding to each of a subset of the collection of learned variants to train a supervised learning framework; and assessing the pathogenicity of the unknown variant based on the trained supervised learning framework.

19. The computer-implemented method of claim 18, further comprising: comparing the set of side information corresponding to each of a subset of the collection of learned variants, wherein the set of side information corresponding to each subsets of the collection of learned variants is compared in relation to similarity scores associated with the subsets of the collection of learned variants.

20. The computer-implemented method of claim 18, further comprising:
assessing the pathogenicity of the unknown variant in relation to the pathogenicity of a nearest variant further comprising:
determining at least one probability for the nearest variant in relation to pathogenic metrics based on a collection of learned variants, wherein the pathogenic metrics comprise a data representation of at least one genetic condition cluster for computing the at least one probability for the nearest variant; and
generating a combined representation of the at least one probability, wherein the combined representation is outputted with respect to the pathogenic metrics.

21. The computer-implemented method of claim 20, further comprising:
at least one of
generating the combined representation by averaging the at least one probability for each variant of a subset of the collection of learned variants, in response to the subset of the collection of learned variants comprise two or more variants with equivalent similarity score such that the nearest variant cannot be determined; and
generating the combined representation using the supervised learning framework based on at least one probability for each variant of a subset of the collection of learned variants given the set of side information, wherein the supervised learning framework comprises one or more supervised prediction models.

22. The computer-implemented method of claim 10, wherein the phenotypic information comprises phenotypic ontology associated with one or more diseases.

23. The computer-implemented method of claim 9, wherein the one or more generative models are configured to decompose the data presentation of annotated data in relation to the pathogenic metrics.

24. The computer-implemented of claim 9, wherein the one or more generative models comprise at least one formulation based on a matrix factorization algorithm.

25. The computer-implemented method of claim 1, wherein the pathogenic metrics comprises at least one classification indicative of a degree of pathogenicity.

26. The computer-implemented method of claim 25, wherein each of the at least one classification is associated with a different optimal set of the at least one genetic condition cluster.

27. A computer-readable medium comprising computer-readable code or instructions stored thereon, which when executed on a processor, causes the processor to implement the computer-implemented method according to claim 1.

28. A system comprising at least one circuitry that is configured to execute the computer-implemented method according to claim 1.

29. An apparatus comprising a processor, a memory and a communication interface, the processor connected to the memory and communication interface, wherein the apparatus is adapted or configured to implement the computer-implemented method according to claim 1.

30. An apparatus for determining pathogenicity of a variant for a patient, the apparatus comprising:
an input component configured to receive the variant;
a processing component configured to determine whether the variant is within a collection of learned variants;
a prediction component, in response to a determination that the variant is present in the collection of the learned variant, configured to generate at least one probability for the variant in relation to pathogenic metrics, wherein the pathogenic metrics comprise a data representation of at least one genetic condition cluster for determining the at least one probability for the variant; and
a display component configured to display the at least one probability for the variant with respect to the pathogenic metrics, wherein the at least one probability is normalised.

31. The apparatus of claim 30, wherein the prediction component, in response to a determination that the variant is absent in the collection of the learned variant, configured to receive a set of side information, wherein the side information is used to identify, in relation to the variant, a nearest variant that is applied as the variant to generate the at least one probability.

32. The apparatus of claim 30, wherein the input component configured to receive phenotypic information associated with the patient, wherein the phenotypic information is applied to adjust the at least one probability for the variant in relation to the at least one genetic condition cluster.

33. A computer-implemented method for determining a probability distribution of pathogenicity for an unknown gene variant using a set of side information, the method comprising:
receiving the unknown variant of a patient, wherein the unknown variant is not identified in or is new to the collection of learned variants associated with a plurality of patients;
assessing the pathogenicity of the unknown gene variant by using a supervised learning framework based on the set of side information; and
determining the probability distribution of pathogenicity based on the assessment.

34. The computer-implemented method of claim 33, further comprising:
computing a probability of the unknown variant associated with a set of pathogenic metrics given the set of side information.

35. The computer-implemented method of claim 33, further comprising:
determining at least one probability for the unknown variant in relation to pathogenic metrics based on a collection of learned variants; and
generating a combined representation of the at least one probability, wherein the combined representation is outputted with respect to the pathogenic metrics.

36. The computer-implemented method of claim 33, wherein the supervised learning framework comprises one or more prediction models.

37. The computer-implemented method of claim 33, wherein the supervised learning framework comprises a non-parametric classifier.

38. The computer-implemented method of claim 33, wherein the set of side information is associated with the unknown gene variant.

39. A computer-readable medium comprising computer-readable code or instructions stored thereon, which when executed on a processor, causes the processor to implement the computer-implemented method of claim 33.

40. The computer-implemented method of claim 2, wherein the phenotypic information comprises phenotypic ontology associated with one or more diseases.

41. An apparatus comprising a processor, a memory and a communication interface, the processor connected to the memory and communication interface, wherein the apparatus is adapted or configured to implement the computer-implemented method according to claim 33.
